SEQUENCE LISTING (5 sequences)

SEQ ID NO: 1 - protein, 568 aa, Homo sapiens
MPSTDLLMLKAFEPYLEILEVYSTKAKNYVNGHCTKYEPWQLIAWSVVWTLLIVWGYEFVFQPESLWSRFKKKCFKLTRKMPIIGRKIQDKLNKTKDDISKNMSFLKVDKEYVKALPSQGLSSSAVLEKLKEYSSMDAFWQEGRASGTVYSGEEKLTELLVKAYGDFAWSNPLHPDIFPGLRKIEAEIVRIACSLFNGGPDSCGCVTSGGTESILMACKAYRDLAFEKGIKTPEIVAPQSAHAAFNKAASYFGMKIVRVPLTKMMEVDVRAMRRAISRNTAMLVCSTPQFPHGVIDPVPEVAKLAVKYKIPLHVDACLGGFLIVFMEKAGYPLEHPFDFRVKGVTSISADTHKYGYAPKGSSLVLYSDKKYRNYQFFVDTDWQGGIYASPTIAGSRPGGISAACWAALMHFGENGYVEATKQIIKTARFLKSELENIKGIFVFGNPQLSVIALGSRDFDIYRLSNLMTAKGWNLNQLQFPPSIHFCITLLHARKRVAIQFLKDIRESVTQIMKNPKAKTTGMGAIYGMAQTTVDRNMVAELSSVFLDSLYSTDTVTQGSQMNGSPKPH

SEQ ID NO: 2 - DNA, 1704 bp, Homo sapiens
  source 1..1704: mol_type = other DNA
atgcctagcacagaccttctgatgttgaaggcctttgagccctacttagagattttggaagtatactccacaaaagccaagaattatgtaaatggacattgcaccaagtatgagccctggcagctaattgcatggagtgtcgtgtggaccctgctgatagtctggggatatgagtttgtcttccagccagagagtttatggtcaaggtttaaaaagaaatgttttaagctcaccaggaagatgcccattattggtcgtaagattcaagacaagttgaacaagaccaaggatgatattagcaagaacatgtcattcctgaaagtggacaaagagtatgtgaaagctttaccctcccagggtctgagctcatctgctgttttggagaaacttaaggagtacagctctatggacgccttctggcaagaggggagagcctctggaacagtgtacagtggggaggagaagctcactgagctccttgtgaaggcttatggagattttgcatggagtaaccccctgcatccagatatcttcccaggactacgcaagatagaggcagaaattgtgaggatagcttgttccctgttcaatgggggaccagattcgtgtggatgtgtgacttctgggggaacagaaagcatactgatggcctgcaaagcatatcgggatctggcctttgagaaggggatcaaaactccagaaattgtggctccccaaagtgcccatgctgcatttaacaaagcagccagttactttgggatgaagattgtgcgggtcccattgacgaagatgatggaggtggatgtgcgggcaatgagaagagctatctccaggaacactgccatgctcgtctgttctaccccacagtttcctcatggtgtaatagatcctgtccctgaagtggccaagctggctgtcaaatacaaaataccccttcatgtcgacgcttgtctgggaggcttcctcatcgtctttatggagaaagcaggatacccactggagcacccatttgatttccgggtgaaaggtgtaaccagcatttcagctgacacccataagtatggctatgccccaaaaggctcatcattggtgttgtatagtgacaagaagtacaggaactatcagttcttcgtcgatacagattggcagggtggcatctatgcttccccaaccatcgcaggctcacggcctggtggcattagcgcagcctgttgggctgccttgatgcacttcggtgagaacggctatgttgaagctaccaaacagatcatcaaaactgctcgcttcctcaagtcagaactggaaaatatcaaaggcatctttgtttttgggaatccccaattgtcagtcattgctctgggatcccgtgattttgacatctaccgactatcaaacctgatgactgctaaggggtggaacttgaaccagttgcagttcccacccagtattcatttctgcatcacattactacacgcccggaaacgagtagctatacaattcctaaaggacattcgagaatctgtcactcaaatcatgaagaatcctaaagcgaagaccacaggaatgggtgccatctatggcatggcccagacaactgttgacaggaatatggttgcagaattgtcctcagtcttcttggacagcttgtacagcaccgacactgtcacccagggcagccagatgaatggttctccaaaaccacac

SEQ ID NO: 3 - DNA, 2101 bp, Homo sapiens
  source 1..2101: mol_type = other DNA
acacacacacacagggcttcatcggctcagagagttgcaaacccagacccatgttctaggctgggctctgccaggaactgctaggagtcgcatctctctctggacctcagtttcttcacgtgtatttgtgtattctctggggtggagctggcgaatagctggagacccctctagccctaccattttttgattctaattaaccaaaaaggatattcgaggtccccgctacaaattctgaacccttggcttcccctccaaactcccacacaaactccaccccatcctgcctgtgtgtctttgggaggatcatttccttctttggtcttggtgtccttgtttacaggttgagggatgataagagtcacctgatctgggcaagtcaggccataaataaggcttgtatgtaaaggtgcctagcatagttcttggcaaagcaaggatcagtcgatatgattgcattggtttgagcatctgggaagaatgagcagactgcatactgtgtgttagggtgtagggaggaatcagacgtgaccctgccctctagtcatctgggtgcacagattctagattaaaggatgtgggggtggtaactaacaaggagatggagaggaggtgtgcagttcctgggggatctgatgaagaattaaagcagaagggtaggagatggagaatgggaaggcatacctgaaaccctaaagcaactcttgatgagtaggcattggcaggaggccccagaacattttctgaccctcaaacactgagaatgcatgtctgctggggaatagtagtgagggggctaagggtatgggggtgttcatgcctgagtcaggggctctggatggtagaaatgtctgggagttctgacggaatgggggtgaggaggcctaaagataacctgttcatagtctttcagggccttaaccattatggctggggaagtgggaagtgccgatggggtatgcagaggagggcagctgtgatagtacatttcatcctgaaagcactacatgtggataatgacggtagtgatgatgccctaattttagtgtcatggcaacattttccaaggggatggaattgggtaccctcattagtgtcatttttgtttgagagatttcaagagtctatctaggtgtcattttaaaatctattgaaaaaaaatcttgaatgcaggtcactgaccagttggttccatgtaactgcactgccttctgctaccactaaagcaatgttatgacaacctggacaagtcatgggaaacctatgtgagacttagttttttctttttctttttggagttgggggtctcactatgttgctcaggctgatctagagctcctgggctcagacaaccctcccgcctcagcatccccaagtgccgggattaccgatgtaagccactacacccagctgagacttagttttttcatctggaaatgaaggcgacctttgtgatcccagtgggcccttgcacttgtgtataaggtgattcccagcactgctcatgaaagtagtgatggcagagtagcctctgctctccattttatttttggggtgggggggggcagtggtggaggggaggatgaggatgatatttccattaaataagtttccttgttgcttttcttcaggcaccactgacctcgatggaaaaatgaagtccctggcccaggaaagagacccaactctgtggctgttttggattagtttgtacaatgccctgcagacctactctctcaggaggctccaagtgaccaaatgtgacaagaaagttttttgtccaaggcagtttggaagagaagttggtgaccgtcgggataccacagccgccccaggacggcctggttgagacattcactggagggtctgggtgcagcccgctgcctggccggtaggcggcgcgcacaggccgtggggcccgggtctgggcgtgcgcgcggctggtagcagcggggccgcgcacgccagggtccgggagccgggccggtgcccccggagccatttccgggaggggcgaggccggcggctgccgggcctccaatctcggcggcggcggcggcaacaggggagcctgggtctcgcggcctgcgagtccgtcgcg

SEQ ID NO: 4 - DNA, 6312 bp, synthetic construct
  source 1..6312: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcgccaccatgcctagcacagaccttctgatgttgaaggcctttgagccctacttagagattttggaagtatactccacaaaagccaagaattatgtaaatggacattgcaccaagtatgagccctggcagctaattgcatggagtgtcgtgtggaccctgctgatagtctggggatatgagtttgtcttccagccagagagtttatggtcaaggtttaaaaagaaatgttttaagctcaccaggaagatgcccattattggtcgtaagattcaagacaagttgaacaagaccaaggatgatattagcaagaacatgtcattcctgaaagtggacaaagagtatgtgaaagctttaccctcccagggtctgagctcatctgctgttttggagaaacttaaggagtacagctctatggacgccttctggcaagaggggagagcctctggaacagtgtacagtggggaggagaagctcactgagctccttgtgaaggcttatggagattttgcatggagtaaccccctgcatccagatatcttcccaggactacgcaagatagaggcagaaattgtgaggatagcttgttccctgttcaatgggggaccagattcgtgtggatgtgtgacttctgggggaacagaaagcatactgatggcctgcaaagcatatcgggatctggcctttgagaaggggatcaaaactccagaaattgtggctccccaaagtgcccatgctgcatttaacaaagcagccagttactttgggatgaagattgtgcgggtcccattgacgaagatgatggaggtggatgtgcgggcaatgagaagagctatctccaggaacactgccatgctcgtctgttctaccccacagtttcctcatggtgtaatagatcctgtccctgaagtggccaagctggctgtcaaatacaaaataccccttcatgtcgacgcttgtctgggaggcttcctcatcgtctttatggagaaagcaggatacccactggagcacccatttgatttccgggtgaaaggtgtaaccagcatttcagctgacacccataagtatggctatgccccaaaaggctcatcattggtgttgtatagtgacaagaagtacaggaactatcagttcttcgtcgatacagattggcagggtggcatctatgcttccccaaccatcgcaggctcacggcctggtggcattagcgcagcctgttgggctgccttgatgcacttcggtgagaacggctatgttgaagctaccaaacagatcatcaaaactgctcgcttcctcaagtcagaactggaaaatatcaaaggcatctttgtttttgggaatccccaattgtcagtcattgctctgggatcccgtgattttgacatctaccgactatcaaacctgatgactgctaaggggtggaacttgaaccagttgcagttcccacccagtattcatttctgcatcacattactacacgcccggaaacgagtagctatacaattcctaaaggacattcgagaatctgtcactcaaatcatgaagaatcctaaagcgaagaccacaggaatgggtgccatctatggcatggcccagacaactgttgacaggaatatggttgcagaattgtcctcagtcttcttggacagcttgtacagcaccgacactgtcacccagggcagccagatgaatggttctccaaaaccccactgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 5 - DNA, 6435 bp, synthetic construct
  source 1..6435: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccggtaccgccaccatggcgagcacagaccttctgatgttgaaggcctttgagccctacttagagattttggaagtatactccacaaaagccaagaattatgtaaatggacattgcaccaagtatgagccctggcagctaattgcatggagtgtcgtgtggaccctgctgatagtctggggatatgagtttgtcttccagccagagagtttatggtcaaggtttaaaaagaaatgttttaagctcaccaggaagatgcccattattggtcgtaagattcaagacaagttgaacaagaccaaggatgatattagcaagaacatgtcattcctgaaagtggacaaagagtatgtgaaagctttaccctcccagggtctgagctcatctgctgttttggagaaacttaaggagtacagctctatggacgccttctggcaagaggggagagcctctggaacagtgtacagtggggaggagaagctcactgagctccttgtgaaggcttatggagattttgcatggagtaaccccctgcatccagatatcttcccaggactacgcaagatagaggcagaaatcgtgaggatagcttgttccctgttcaatgggggaccagattcgtgtggatgtgtgacttctgggggaacagaaagcatactgatggcctgcaaagcatatcgggatctggcctttgagaaggggatcaaaactccagaaattgtggctccccaaagtgcccatgctgcatttaacaaagcagccagttactttgggatgaagattgtgcgggtcccattgacgaagatgatggaggtggatgtgcgggcaatgagaagagctatctccaggaacactgccatgctcgtctgttctaccccacagtttcctcatggtgtaatagatcctgtccctgaagtggccaagctggctgtcaaatacaaaataccccttcatgtcgacgcttgtctgggaggcttcctcatcgtctttatggagaaagcaggatacccactggagcacccatttgatttccgggtgaaaggtgtaaccagcatttcagctgacacccataagtatggctatgccccaaaaggctcatcattggtgttgtatagtgacaagaagtacaggaactatcagttcttcgtcgatacagattggcagggtggcatctatgcttccccaaccatcgcaggctcacggcctggtggcattagcgcagcctgttgggctgccttgatgcacttcggtgagaacggctatgttgaagctaccaaacagatcatcaaaactgctcgcttcctcaagtcagaactggaaaatatcaaaggcatctttgtttttgggaatccccaattgtcagtcattgctctgggatcccgtgattttgacatctaccgactatcaaacctgatgactgctaaggggtggaacttgaaccagttgcagttcccacccagtattcatttctgcatcacattactacacgcccggaaacgagtagctatacaattcctaaaggacattcgagaatctgtcactcaaatcatgaagaatcctaaagcgaagaccacaggaatgggtgccatctatggcatggcccagacaactgttgacaggaatatggttgcagaattgtcctcagtcttcttggacagcttgtacagcaccgacactgtcacccagggcagccagatgaatggttctccaaaaccccactgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccag

(19) United States
(12) Patent Application Publication
(10) Pub. No.: US 2025/0177563 A1
(43) Pub. Date: Jun. 5, 2025

(54) Adeno-Associated Viral (AAV)-Mediated Sphingosine-1-Phosphate Lyase (SPL) Expression for Treating Pulmonary Fibrosis

(71) Applicant: The Regents of the University of California, Oakland, CA (US)

(72) Inventors: Julie D. Saba, Oakland, CA (US); Mallar Bhattacharya, San Francisco, CA (US)

(21) Appl. No.: 18/293,302

(22) PCT Filed: Aug. 2, 2022
(86) PCT No.: PCT/US2022/074437

§ 371 Date: Jan. 29, 2024

Related U.S. Application Data
(60) Provisional application No. 63/228,839, filed on Aug. 3, 2021.

(51) Int. Cl. A61K 48/00; A61K 31/4418; A61K 31/496; A61K 38/51; A61P 11/00; C12N 15/86

(52) U.S. Cl. CPC A61K 48/005; A61K 31/4418; A61K 31/496; A61K 38/51; A61K 48/0075; A61P 11/00; C12N 15/86; C12Y 401/02027; C12N 2750/14143

(57) ABSTRACT

The present disclosure provides methods treating or preventing pulmonary fibrosis in a subject in need thereof by administering to the subject a therapeutically effective dose of a recombinant adeno-associated viral (rAAV) virion comprising a nucleic acid encoding sphingosine-1-phosphate lyase (SPL), where administering the rAAV virion results in expression of the SPL in the subject thereby treating or preventing the pulmonary fibrosis in the subject.

### CROSS-REFERENCE TO RELATED APPLICATION

This application claims priority to U.S. Provisional Application Ser. No. 63/228,839, filed on Aug. 3, 2021, the disclosure of which is herein incorporated by reference in its entirety.

### INTRODUCTION

Pulmonary fibrosis is a severe, relentless and progressive lung disease that may be idiopathic or may be associated with risk factors including gastroesophageal reflux, smoking, asbestos, grain dust, hard metal, dust, various types of chemotherapy and other medications, radiation and diseases such as autoimmune diseases and sarcoidosis. It is the result of lung tissue damage and scarring that leads to symptoms of shortness of breath, cough, fatigue, weight loss, and muscle aching. It can have severe, acute exacerbations that can lead to hospitalizations, artificial ventilation requirement, and death. Approximately 130,000 persons in the USA have idiopathic pulmonary fibrosis, with 30-40 thousand new cases annually. 3 million persons worldwide have idiopathic pulmonary fibrosis. Up to 33% of survivors of coronavirus infections develop pulmonary fibrosis, with an observed rate of 4.9% in survivors of the ongoing SARS-CoV-2 pandemic. This would predict >10 million cases of pulmonary fibrosis may develop in coming years. The disease carries additional risk of cardiovascular disease, pulmonary hypertension, lung cancer, lung complications, right heart failure, and venous thromboembolic disease. While there are some anti-inflammatory and anti-fibrotic drugs to treat the disease, it can progress despite these treatments.

SGPL1 gene encodes the vitamin B6-dependent enzyme sphingosine-1-phosphate lyase (SPL, also known as, S1PL), which catalyzes the irreversible degradation of the bioactive sphingolipid sphingosine-1-phosphate (S1P) in the final step of sphingolipid degradation (Expert Opin Ther Targets. 2009; 13 (8): 1013-25; J Lipid Res. 2019; 60 (3): 456-63). S1P serves as a ligand for a family of ubiquitously expressed G protein-coupled receptors (S1PRs). The S1PRs control actin cytoskeleton organization, cell migration, morphology and cell survival by signaling through downstream targets including MAPK, AKT and Rho GTPases (J Recept Signal Transduct Res. 2017; 37 (5): 437-46). S1P is generated from sphingosine by sphingosine kinases (SphK1/2) via a phosphorylation event that can be reversed by lipid- and S1P-specific phosphatases (Sgpp1/2) (Biochim Biophys Acta Mol Cell Biol Lipids. 2018; 1863 (11): 1413-22; J Lipid Res. 2015; 56 (11): 2048-60). However, only SPL can irreversibly degrade S1P. Thus, SPL is a critical regulator of S1P levels in blood and tissues (Expert Opin Ther Targets. 2009; 13 (8): 1013-25).

### SUMMARY

The present disclosure provides methods for treating pulmonary fibrosis in a subject, the method comprising administering to the subject a therapeutically effective dose of a recombinant adeno-associated viral (rAAV) virion comprising a nucleic acid encoding sphingosine-1-phosphate lyase (SPL), wherein administering the rAAV virion results in expression of the SPL in the subject thereby treating the pulmonary fibrosis in the subject.

The present disclosure provides methods for preventing pulmonary fibrosis in a subject, the method comprising administering to the subject a therapeutically effective dose of a recombinant adeno-associated viral (rAAV) virion comprising a nucleic acid encoding sphingosine-1-phosphate lyase (SPL), wherein administering the rAAV virion results in expression of the SPL in the subject thereby preventing the pulmonary fibrosis in the subject.

### BRIEF DESCRIPTION OF THE DRAWINGS

Included in the drawings are the following FIGURES:

FIG. 1. AAV-SPL protects against bleomycin-induced pulmonary fibrosis.

Before exemplary embodiments of the present invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a rAAV virion” includes a plurality of such rAAV virions and reference to “the vector” includes reference to one or more vectors, “a mutation” refers to one or more mutations, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflicts with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

“AAV” is an abbreviation for adeno-associated virus, and may be used to refer to the virus itself or derivatives thereof, such as, engineered AAVs. The term covers all subtypes and both naturally occurring and recombinant forms, except where required otherwise. The abbreviation “rAAV” refers to recombinant adeno-associated virus, also referred to as a recombinant AAV vector (or “rAAV vector”). The term “AAV” includes all AAV serotypes as well as AAV vectors based on the combination of different serotypes (also referred to as “hybrid AAV vectors” or “pseudotype AAV vectors”). In one aspect, the AAV vector is an AAV Vector with a serotype selected from AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), avian AAV, bovine AAV, canine AAV, equine AAV, primate AAV, non-primate AAV, ovine AAV, AAV-7m8 and combinations thereof. See, e.g., Mori et al. (2004) Virology 330:375. The synthetic AAV variant AAV-7m8 is described, for example, in Dalkara et al. See Transl Med 2013, 5 (189): 189ra76. The term “AAV” also includes chimeric AAV. “Primate AAV” refers to AAV isolated from a primate, “non-primate AAV” refers to AAV isolated from a non-primate mammal, “bovine AAV” refers to AAV isolated from a bovine mammal (e.g., a cow), etc. AAV also encompasses mixed serotype capsids, AAV-PHP.B, AAV-PHP.B2, AAV-PHP.B3, AAV-PHP.A, AAV-PHP.eB, AAV-PHP.eS, evolved capsids that are less immunogenic to mice and humans, and variants thereof.

An “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two AAV inverted terminal repeat sequences (ITRs). The term rAAV vector encompasses both rAAV vector particles and rAAV vector plasmids.

An “AAV virus” or “AAV viral particle” or “rAAV vector particle” refers to a viral particle composed of at least one AAV capsid protein (typically by all of the capsid proteins of a wild-type AAV) and an encapsidated polynucleotide rAAV vector. If the particle comprises a heterologous polynucleotide (i.e. a polynucleotide other than a wild-type AAV genome, such as a transgene to be delivered to a mammalian cell), it is typically referred to as an “rAAV vector particle” or simply an “rAAV vector”. Thus, production of rAAV particle necessarily includes production of rAAV vector, as such a vector is contained within an rAAV particle.

“Packaging” refers to a series of intracellular events that result in the assembly and encapsidation of an AAV particle.

AAV “rep” and “cap” genes refer to polynucleotide sequences encoding replication and encapsidation proteins of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). “Helper virus function” may be provided in a number of ways, including by providing helper virus or providing, for example, polynucleotide sequences encoding the requisite function(s) to a producer cell in trans.

An “infectious” virus or viral particle is one that comprises a polynucleotide component which it is capable of delivering into a cell for which the viral species is tropic. The term does not necessarily imply any replication capacity of the virus. As used herein, an “infectious” virus or viral particle is one that can access a target cell, can infect a target cell, and can express a heterologous nucleic acid in a target cell. Thus, “infectivity” refers to the ability of a viral particle to access a target cell, infect a target cell, and express a heterologous nucleic acid in a target cell. Infectivity can refer to in vitro infectivity or in vivo infectivity. Assays for counting infectious viral particles are described elsewhere in this disclosure and in the art. Viral infectivity can be expressed as the ratio of infectious viral particles to total viral particles. Total viral particles can be expressed as the number of viral genome (vg) copies. The ability of a viral particle to express a heterologous nucleic acid in a cell can be referred to as “transduction.” The ability of a viral particle to express a heterologous nucleic acid in a cell can be assayed using a number of techniques, including assessment of a marker gene, such as a green fluorescent protein (GFP) assay (e.g., where the virus comprises a nucleotide sequence encoding GFP), where GFP is produced in a cell infected with the viral particle and is detected and/or measured; or the measurement of a produced protein, for example by an enzyme-linked immunosorbent assay (ELISA). Viral infectivity can be expressed as the ratio of infectious viral particles to total viral particles. Methods of determining the ratio of infectious viral particle to total viral particle are known in the art.

A “replication-competent” virus (e.g. a replication-competent AAV) refers to a phenotypically wild-type virus that is infectious, and is also capable of being replicated in an infected cell (i.e. in the presence of a helper virus or helper virus functions). In the case of AAV, replication competence generally requires the presence of functional AAV packaging genes. In general, rAAV vectors as described herein are replication-incompetent in mammalian cells (especially in human cells) by virtue of the lack of one or more AAV packaging genes. Typically, such rAAV vectors lack any AAV packaging gene sequences in order to minimize the possibility that replication competent AAV are generated by recombination between AAV packaging genes and an incoming rAAV vector. In many embodiments, rAAV vector preparations as described herein are those which contain few if any replication competent AAV (rcAAV, also referred to as RCA) (e.g., less than about 1 rcAAV per 10² rAAV particles, less than about 1 rcAAV per 10⁴ rAAV particles, less than about 1 rcAAV per 10⁸ rAAV particles, less than about 1 rcAAV per 10¹² rAAV particles, or no rcAAV).

The term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST or FASTA. Of particular interest are alignment programs that permit gaps in the sequence. Of interest is the BestFit program using the local homology algorithm of Smith Waterman (Advances in Applied Mathematics 2:482-489 (1981) to determine sequence identity.

A “gene” refers to a polynucleotide containing at least one open reading frame that is capable of encoding a particular protein after being transcribed and translated.

“Recombinant,” as applied to a polynucleotide means that the polynucleotide is the product of various combinations of cloning, restriction or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

An “expression vector” is a vector comprising a region which encodes a polypeptide of interest, and is used for effecting the expression of the protein in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

“Heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter. Thus, for example, an rAAV that includes a heterologous nucleic acid encoding a heterologous gene product is an rAAV that includes a nucleic acid not normally included in a naturally-occurring, wild-type AAV, and the encoded heterologous gene product is a gene product not normally encoded by a naturally-occurring, wild-type AAV.

As used herein, the terms “treatment,” “treating,” and the like, refer to obtaining a desired pharmacologic and/or physiologic effect. The effect may be prophylactic in terms of completely or partially preventing a onset of a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: a) inhibiting the disease, i.e., arresting or slowing its development; and (b) relieving the disease, i.e., causing regression of the disease. One form of treatment includes prevention of occurrence of a disease or delay in onset of the disease and/or reduced severity of the disease after onset of the disease in a subject which may be predisposed to the disease or at risk of acquiring the disease but has not yet been diagnosed as having it.

The terms “individual,” “host,” “subject,” and “patient” are used interchangeably herein, and refer to a mammal, including, but not limited to, human and non-human primates, including simians and humans; mammalian sport animals (e.g., horses, camels, etc.); mammalian farm animals (e.g., sheep, goats, cows, etc.); mammalian pets (dogs, cats, etc.); and rodents (e.g., mice, rats, etc.). In some cases, the individual is a human.

The term “pharmaceutically acceptable” refers to a non-toxic material which preferably does not interfere with the action of the active ingredient of the pharmaceutical composition. In particular, the term “pharmaceutically acceptable” means that the subject substance has been approved by a governmental regulatory agency for use in animals, and particularly humans, or in U.S. Pat. Pharmacopoeia, European Pharmacopoeia or other recognized pharmacopoeias for use in animals and in particular humans.

### Methods

The present disclosure provides a method for treating pulmonary fibrosis in a subject. The method involves administering to the subject a therapeutically effective dose of a recombinant adeno-associated viral (rAAV) virion comprising a nucleic acid encoding sphingosine-1-phosphate lyase (SPL), wherein administering the rAAV virion results in expression of the SPL in the subject thereby treating the pulmonary fibrosis in the subject. The present disclosure also encompasses prevention of onset of pulmonary fibrosis in a subject at risk for developing pulmonary fibrosis. The method involves administering to the subject a therapeutically effective dose of a recombinant adeno-associated viral (rAAV) virion comprising a nucleic acid encoding sphingosine-1-phosphate lyase (SPL), wherein administering the rAAV virion results in expression of the SPL in the subject thereby preventing onset of the pulmonary fibrosis in the subject. Various steps and aspects of the method will now be described in greater detail below.

SGPL1 gene encodes the vitamin B6-dependent enzyme SPL, which catalyzes the irreversible degradation of the bioactive sphingolipid sphingosine-1-phosphate (S1P) in the final step of sphingolipid degradation.

A subject who may be treated with the methods disclosed herein may be a human, e.g., an adult, a teen, or a child diagnosed as having pulmonary fibrosis. A subject who may be administered the rAAV virion disclosed herein for preventing onset of pulmonary fibrosis may be a human, e.g., a child, a teenager, or an adult at risk for developing pulmonary fibrosis. Risk factors that can lead to onset of pulmonary fibrosis can include gastroesophageal reflux, smoking, exposure to asbestos, grain dust, hard metal, dust, various types of chemotherapy and other medications, radiation and diseases such as autoimmune diseases and sarcoidosis.

In certain aspects, the subject receiving the rAAV virion is not a subject having S1P lyase insufficiency syndrome (SPLIS). For example, the subject does not harbor inactivating mutations in SGPL1 gene and/or does not exhibit one or more major SPLIS disease features including steroid-resistant nephrotic (protein spilling) syndrome with focal segmental glomerulosclerosis (FSGS) pathology, neurological defects (developmental delay or regression, ataxia, cranial nerve defects, seizures, and peripheral neuropathy), ichthyosis, cranial nerve palsies, bony abnormalities, hypocalcemia, hypothyroidism, gonadal defects, lymphopenia/immunodeficiency and primary adrenal insufficiency with a wide range of severity. In other aspects, the subject receiving the rAAV virion is a SPLIS patient. SPL inactivating mutations are known. An SPL inactivating mutation may be homozygous or heterozygous. In certain aspects, the SPL inactivating mutation may be a frameshift mutation, truncation, a mutation resulting in splicing defect, a substitution, deletion, mutation in 5′ untranslated region of the gene that prevent expression, mutations in introns, exons, or 3′-untranslated region of the mRNA that promote mRNA degradation. In certain aspects, the mutation may be a frameshift mutation (e.g., Gly360Alafs*49, Ser3Lysfs*11, Ser65Argfs*6, Arg278Glyfs*17, Leu312Phefs*30, or Phe411Leufs*56,), a truncation (e.g., Tyr331*, Ser361*, or Arg505*), a mutation resulting in splicing defect, a substitution, e.g., a substitution at residue 353, replacing the cofactor binding lysine with an arginine (Lys353Arg), a Arg222Gln substitution, a Gly360Val substitution, a Phe290Leu substitution, a Ser202Leu substitution, a Tyr416Cys substitution, a Cys285Tyr substitution, a Tyr15Cys substitution, a Lys353Arg substitution, a Ile184Thr substitution, a Arg340Trp substitution, a Ser346Ile substitution, or a combination thereof. In certain aspects, a subject may have more than one mutation in the SPL gene.

The rAAV virion may be administered in a therapeutically effective amount. A “therapeutically effective amount” will fall in a relatively broad range that can be determined through experimentation and/or clinical trials. For example, a therapeutically effective dose can be on the order of from about 10⁶ to about 10¹⁵ of the rAAV virions/dose, e.g., from about 10⁸ to 10¹² rAAV virions, from about 10⁶ viral genomes (vg) to about 10¹⁵ vg of the rAAV virions, e.g., from about 10⁸ vg to 10¹² vg per dose. Other effective dosages can be readily established by one of ordinary skill in the art through routine trials establishing dose response curves.

In some embodiments, more than one administration (e.g., two, three, four or more administrations) may be employed to achieve the desired level of gene expression. In some cases, the more than one administration is administered at various intervals, e.g., daily, weekly, twice monthly, monthly, every 3 months, every 6 months, yearly, etc. In some cases, multiple administrations are administered over a period of time from 1 month to 2 months, from 2 months to 4 months, from 4 months to 8 months, from 8 months to 12 months, from 1 year to 2 years, from 2 years to 5 years, or more than 5 years.

In some embodiments the AAV serotype is selected from: AAV1, AAV2, AAV3, AAV4, AAV5, AAV6, AAV7, AAV8, and AAVrh.10. In other embodiments the AAV serotype is a variant of an AAV serotype is selected from: AAV1, AAV2, AAV3, AAV4, AAV5, AAV6, AAV7, AAV8, and AAVrh.10. In some aspects, the rAAV virion comprises AAV serotype 9 capsid proteins. The nucleic acid encoding SPL is flanked by AAV inverted terminal repeats (ITRs), e.g., AAV2 ITRs or AAV9 ITRs.

In certain aspects, the subject may be human and the SPL may be a human SPL. The nucleic acid present in the AAV may encode a human SPL having an amino acid sequence that is at least 80%, 85%, 90%, 95%, 96%, 97%, 98%, or 99% or a 100% identical to the amino acid sequence:

(SEQ ID NO: 1)
MPSTDLLMLKAFEPYLEILEVYSTKAKNYVNGHCTKYEPWQLIAWSVVW
TLLIVWGYEFVFQPESLWSRFKKKCFKLTRKMPIIGRKIQDKLNKTKDD
ISKNMSFLKVDKEYVKALPSQGLSSSAVLEKLKEYSSMDAFWQEGRASG
TVYSGEEKLTELLVKAYGDFAWSNPLHPDIFPGLRKIEAEIVRIACSLE
NGGPDSCGCVTSGGTESILMACKAYRDLAFEKGIKTPEIVAPQSAHAAF
NKAASYFGMKIVRVPLTKMMEVDVRAMRRAISRNTAMLVCSTPQFPHGV
IDPVPEVAKLAVKYKIPLHVDACLGGFLIVFMEKAGYPLEHPFDFRVKG
VTSISADTHKYGYAPKGSSLVLYSDKKYRNYQFFVDTDWQGGIYASPTI
AGSRPGGISAACWAALMHFGENGYVEATKQIIKTARFLKSELENIKGIF
VFGNPQLSVIALGSRDFDIYRLSNLMTAKGWNLNQLQFPPSIHFCITLL
HARKRVAIQFLKDIRESVTQIMKNPKAKTTGMGAIYGMAQTTVDRNMVA
ELSSVFLDSLYSTDTVTQGSQMNGSPKPH.

As used herein, human SPL refers to a polypeptide having the amino acid sequence set forth above as SEQ ID NO:1, a functional fragment thereof, or a polypeptide having SPL activity and having an amino acid sequence at least 80% identical (e.g., at least 85%, at least 90%, at least 95%, at least 96%, at least 97%, at least 98%, or at least 99%) to the amino acid sequence set forth in SEQ ID NO:1. In certain aspects, the nucleic acid encoding the human SPL may be a codon-optimized sequence, where the codons are codons from highly expressed human genes.

In certain aspects, the SPL may be a soluble human SPL that lacks the transmembrane region of the full-length SPL. For example, the soluble human SPL may lack an N-terminal sequence, such as, the first 66 N-terminal amino acids present in SEQ ID NO:1.

Treatment of pulmonary fibrosis in a subject in need thereof may result in a decrease of lung tissue damage and/or scarring in the subject. The decrease may be at least 10% or more (e.g., 20%, 30%, 40%, 50%, or more) as compared to prior to the treatment. The treatment may increase life span of the subject by at least 1 year or more, e.g., at least 5 years, 10 years, 15 years, or more as compared to subjects with pulmonary fibrosis not receiving the treatment.

### Recombinant AAVs: Production Methods

Methods for obtaining recombinant AAVs having a desired capsid protein are well known in the art. (See, for example, US 2003/0138772, the contents of which are incorporated herein by reference in their entirety). Typically, the methods involve culturing a host cell which contains a nucleic acid sequence encoding an AAV capsid protein or fragment thereof; a functional rep gene; a recombinant AAV vector composed of, AAV inverted terminal repeats (ITRs) and a therapeutic transgene; and sufficient helper functions to permit packaging of the recombinant AAV vector into the AAV capsid proteins.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the invention may be delivered to the packaging host cell using any appropriate genetic element (vector). The selected genetic element may be delivered by any suitable method, including those described herein. The methods used to construct any embodiment of this invention are known to those with skill in nucleic acid manipulation and include genetic engineering, recombinant engineering, and synthetic techniques.

Typically, the recombinant AAVs are produced by transfecting a host cell with an recombinant AAV vector (comprising a therapeutic transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpes virus (other than herpes simplex virus type-1), and vaccinia virus.

A “host cell” refers to any cell that harbors, or is capable of harboring, a substance of interest. Often a host cell is a mammalian cell. A host cell may be used as a recipient of an AAV helper construct, an AAV transgene plasmid, e.g., comprising a promoter operably linked with a S1PL encoding gene, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter.

The foregoing methods for packaging recombinant vectors in desired AAV capsids to produce the rAAVs of the present disclosure are not meant to be limiting and other suitable methods will be apparent to the skilled artisan.

### Recombinant AAV Vectors

“Recombinant AAV (rAAV) vectors” of the present disclosure are typically composed of, at a minimum, a therapeutic transgene, e.g., encoding a SPL, and its regulatory sequences, and 5′ and 3′ AAV inverted terminal repeats (ITRs). It is this recombinant AAV vector which is packaged into a capsid protein and delivered to a subject. The nucleic acid sequence encoding a S1PL is operatively linked to regulatory components in a manner which permits transgene transcription, translation, and/or expression in a cell.

The nucleic acid encoding a SPL may have the following sequence or may have a sequence at least 50% identical (e.g., at least 60%, at least 70%, at least 80%, at least 85%, at least 90%, or at least 95% identical) to the following nucleic acid sequence:

(SEQ ID NO: 2)
ATGCCTAGCACAGACCTTCTGATGTTGAAGGCCTTTGAGCCCTACTTA
GAGATTTTGGAAGTATACTCCACAAAAGCCAAGAATTATGTAAATGGAC
ATTGCACCAAGTATGAGCCCTGGCAGCTAATTGCATGGAGTGTCGTGTG
GACCCTGCTGATAGTCTGGGGATATGAGTTTGTCTTCCAGCCAGAGAGT
TTATGGTCAAGGTTTAAAAAGAAATGTTTTAAGCTCACCAGGAAGATGC
CCATTATTGGTCGTAAGATTCAAGACAAGTTGAACAAGACCAAGGATGA
TATTAGCAAGAACATGTCATTCCTGAAAGTGGACAAAGAGTATGTGAAA
GCTTTACCCTCCCAGGGTCTGAGCTCATCTGCTGTTTTGGAGAAACTTA
AGGAGTACAGCTCTATGGACGCCTTCTGGCAAGAGGGGAGAGCCTCTGG
AACAGTGTACAGTGGGGAGGAGAAGCTCACTGAGCTCCTTGTGAAGGCT
TATGGAGATTTTGCATGGAGTAACCCCCTGCATCCAGATATCTTCCCAG
GACTACGCAAGATAGAGGCAGAAATTGTGAGGATAGCTTGTTCCCTGTT
CAATGGGGGACCAGATTCGTGTGGATGTGTGACTTCTGGGGGAACAGAA
AGCATACTGATGGCCTGCAAAGCATATCGGGATCTGGCCTTTGAGAAGG
GGATCAAAACTCCAGAAATTGTGGCTCCCCAAAGTGCCCATGCTGCATT
TAACAAAGCAGCCAGTTACTTTGGGATGAAGATTGTGCGGGTCCCATTG
ACGAAGATGATGGAGGTGGATGTGCGGGCAATGAGAAGAGCTATCTCCA
GGAACACTGCCATGCTCGTCTGTTCTACCCCACAGTTTCCTCATGGTGT
AATAGATCCTGTCCCTGAAGTGGCCAAGCTGGCTGTCAAATACAAAATA
CCCCTTCATGTCGACGCTTGTCTGGGAGGCTTCCTCATCGTCTTTATGG
AGAAAGCAGGATACCCACTGGAGCACCCATTTGATTTCCGGGTGAAAGG
TGTAACCAGCATTTCAGCTGACACCCATAAGTATGGCTATGCCCCAAAA
GGCTCATCATTGGTGTTGTATAGTGACAAGAAGTACAGGAACTATCAGT
TCTTCGTCGATACAGATTGGCAGGGTGGCATCTATGCTTCCCCAACCAT
CGCAGGCTCACGGCCTGGTGGCATTAGCGCAGCCTGTTGGGCTGCCTTG
ATGCACTTCGGTGAGAACGGCTATGTTGAAGCTACCAAACAGATCATCA
AAACTGCTCGCTTCCTCAAGTCAGAACTGGAAAATATCAAAGGCATCTT
TGTTTTTGGGAATCCCCAATTGTCAGTCATTGCTCTGGGATCCCGTGAT
TTTGACATCTACCGACTATCAAACCTGATGACTGCTAAGGGGTGGAACT
TGAACCAGTTGCAGTTCCCACCCAGTATTCATTTCTGCATCACATTACT
ACACGCCCGGAAACGAGTAGCTATACAATTCCTAAAGGACATTCGAGAA
TCTGTCACTCAAATCATGAAGAATCCTAAAGCGAAGACCACAGGAATGG
GTGCCATCTATGGCATGGCCCAGACAACTGTTGACAGGAATATGGTTGC
AGAATTGTCCTCAGTCTTCTTGGACAGCTTGTACAGCACCGACACTGTC
ACCCAGGGCAGCCAGATGAATGGTTCTCCAAAACCACAC

In some cases, the nucleic acid encoding a human SPL may be a codon optimized version of the nucleotide sequence set forth in SEQ ID NO:2. For example, one or more codons in SEQ ID NO:2 may be replaced with codon(s) from highly expressed human genes to increase expression of the SPL in a human subject receiving the rAAV.

The AAV sequences of the vector typically comprise the cis-acting 5′ and 3′ inverted terminal repeat sequences. The ITR sequences are about 145 bp in length. An example of such a molecule is a “cis-acting” plasmid containing the nucleic acid sequence encoding a S1PL, in which the nucleic acid sequence encoding a S1PL and associated regulatory elements are flanked by the 5′ and 3′ AAV ITR sequences. The AAV ITR sequences may be obtained from any known AAV, including presently identified mammalian AAV types. The 5′ and 3′ AAV ITR sequences may be AAV9 5′ and 3′ ITR sequences, respectively, or AAV2 5′ and 3′ ITR sequences, respectively.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art.

In certain aspects, the promoter may be a CMV, a chicken beta actin promoter, human β-actin/CMV hybrid promoter, chicken β-actin/CMV hybrid promoter, CMV-actin-globin (CAG) hybrid promoter, Math Promoter, VGLUT3 promoter, parvalbumin promoter, calretinin promoter, calbindin 28 k promoter, prestin promoter, a liver specific promoter, e.g., albumin promoter, endogenous SGPL1 promoter for human SPL, and the like. In certain aspects, the human SGPL1 promoter may have a nucleotide sequence that extends from −200 to +100 of the 5′ region of the SGPL1 gene and has the following sequence:

(SEQ ID NO: 3)
acacacacacacagggcttcatcggctcagagagttgcaaacccagacc
catgttctaggctgggctctgccaggaactgctaggagtcgcatctctc
tctggacctcagtttcttcacgtgtatttgtgtattctctggggtggag
ctggcgaatagctggagacccctctagccctaccattttttgattctaa
ttaaccaaaaaggatattcgaggtccccgctacaaattctgaacccttg
gcttcccctccaaactcccacacaaactccaccccatcctgcctgtgtg
tctttgggaggatcatttccttctttggtcttggtgtccttgtttacag
gttgagggatgataagagtcacctgatctgggcaagtcaggccataaat
aaggcttgtatgtaaaggtgcctagcatagttcttggcaaagcaaggat
cagtcgatatgattgcattggtttgagcatctgggaagaatgagcagac
tgcatactgtgtgttagggtgtagggaggaatcagacgtgaccctgccc
tctagtcatctgggtgcacagattctagattaaaggatgtgggggtggt
aactaacaaggagatggagaggaggtgtgcagttcctgggggatctgat
gaagaattaaagcagaagggtaggagatggagaatgggaaggcatacct
gaaaccctaaagcaactcttgatgagtaggcattggcaggaggccccag
aacattttctgaccctcaaacactgagaatgcatgtctgctggggaata
gtagtgagggggctaagggtatgggggtgttcatgcctgagtcaggggc
tctggatggtagaaatgtctgggagttctgacggaatgggggtgaggag
gcctaaagataacctgttcatagtctttcagggccttaaccattatggc
tggggaagtgggaagtgccgatggggtatgcagaggagggcagctgtga
tagtacatttcatcctgaaagcactacatgtggataatgacggtagtga
tgatgccctaattttagtgtcatggcaacattttccaaggggatggaat
tgggtaccctcattagtgtcatttttgtttgagagatttcaagagtcta
tctaggtgtcattttaaaatctattgaaaaaaaatcttgaatgcaggtc
actgaccagttggttccatgtaactgcactgccttctgctaccactaaa
gcaatgttatgacaacctggacaagtcatgggaaacctatgtgagactt
agttttttctttttctttttggagttgggggtctcactatgttgctcag
gctgatctagagctcctgggctcagacaaccctcccgcctcagcatccc
caagtgccgggattaccgatgtaagccactacacccagctgagacttag
ttttttcatctggaaatgaaggcgacctttgtgatcccagtgggccctt
gcacttgtgtataaggtgattcccagcactgctcatgaaagtagtgatg
gcagagtagcctctgctctccattttatttttggggtgggggggggcag
tggtggaggggaggatgaggatgatatttccattaaataagtttccttg
ttgcttttcttcaggcaccactgacctcgatggaaaaatgaagtccctg
gcccaggaaagagacccaactctgtggctgttttggattagtttgtaca
atgccctgcagacctactctctcaggaggctccaagtgaccaaatgtga
caagaaagttttttgtccaaggcagtttggaagagaagttggtgaccgt
cgggataccacagccgccccaggacggcctggttgagacattcactgga
gggtctgggtgcagcccgctgcctggccggtaggcggcgcgcacaggcc
gtggggcccgggtctgggcgtgcgcgcggctggtagcagcggggccgcg
cacgccagggtccgggagccgggccggtgcccccggagccATTTCCGGG
AGGGGCGAGGCCGGCGGCTGCCGGGCCTCCAATCTCGGCGGCGGCGGCG
GCAACAGGGGAGCCTGGGTCTCGCGGCCTGCGAGTCCGTCGCG.

Sequence from −2000 to +100 where +1 is the start of transcription and sequence upstream of +1 is in lower case. Longer and shorter versions of the human SGPL1 promoter may also be used.

In certain aspects, the AAV vector is selected from the group consisting of AAV-PHP.B, AAV-PHP.eB, AAV-PHP.S and AAV-Anc80. In certain aspects, the AAV vector AAV-PHP.eB or AAV-PHP.S.

In certain aspects, the AAV vector is selected from the group consisting of AAV-8, AAV-9 and AAV-1/2.

### Recombinant AAV Virion Administration Methods

The rAAV virion may be delivered to a subject in compositions according to any appropriate methods known in the art. The rAAV virion, preferably suspended in a physiologically compatible carrier or a pharmaceutically acceptable excipient (i.e., in a composition), may be administered to a subject, such as, for example, a human, mouse, rat, cat, dog, sheep, rabbit, horse, cow, goat, pig, guinea pig, hamster, chicken, turkey, or a non-human primate (e.g., Macaque).

The rAAV virions are administered in sufficient amounts to transfect the cells present in lungs and to provide sufficient levels of gene transfer and expression without undue adverse effects. Conventional and pharmaceutically acceptable routes of administration include, but are not limited to, direct delivery to the lungs, e.g., inhalation (including intranasal and intratracheal delivery), or intravenous, intramuscular, subcutaneous, intradermal, intrathecal, and other parental routes of administration. In certain circumstances it will be desirable to deliver the rAAV-based therapeutic constructs in suitably formulated pharmaceutical compositions disclosed herein either subcutaneously, intranasally, parenterally, intravenously, intramuscularly, orally, intraperitoneally, or by inhalation.

Delivery of the rAAV virions to a mammalian subject may be by intravenous injection. In some embodiments, the mode of administration of rAAV virions is by portal vein injection. Administration into the bloodstream may be by injection into a vein, an artery, or any other vascular conduit. In some embodiments, administration of rAAV virions into the bloodstream is by way of isolated limb perfusion, a technique well known in the surgical arts, the method essentially enabling the artisan to isolate a limb from the systemic circulation prior to administration of the rAAV virions. Routes of administration may be combined, if desired.

The compositions of the invention may comprise a rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes. In certain aspects, the composition may include an additional therapeutic agent for treatment for pulmonary fibrosis.

In a further aspect, the present invention provides a viral vector or a virion of the present invention or a pharmaceutical composition of the present invention for use as a medicament.

In a further aspect, the present invention provides a viral vector or a virion of the present invention or a pharmaceutical composition of the present invention for use in a method of treating pulmonary fibrosis or preventing pulmonary fibrosis.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. Optionally, the compositions of the present disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene expression required to achieve a therapeutic effect, the specific disease or disorder being treated, and the stability of the gene or RNA product. One of skill in the art can readily determine a rAAV virion dose range to treat a subject having a particular disease or disorder based on the aforementioned factors, as well as other factors that are well known in the art.

An effective amount of a rAAV is an amount sufficient to infect a target tissue. The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁶ genome copies. In some cases, a dosage between about 10¹¹ to 10¹² rAAV genome copies is appropriate. In certain embodiments, the dosage of rAAV is 10¹⁰, 10¹¹, 10¹², 10¹³, or 10¹⁴ genome copies per kg. In certain embodiments, the dosage of rAAV is 10¹⁰, 10¹¹, 10¹², 10¹³, 10¹⁴, or 10¹⁵ genome copies per subject.

In some embodiments, rAAV compositions are formulated to reduce aggregation of AAV particles in the composition, particularly where high rAAV concentrations are present (e.g., ˜10¹³ GC/ml or more). Methods for reducing aggregation of rAAVs are well known in the art and, include, for example, addition of surfactants, pH adjustment, salt concentration adjustment, etc.

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the host. The person responsible for administration will, in any event, determine the appropriate dose for the individual host.

Sterile injectable solutions are prepared by incorporating the active rAAV virions in the required amount in the appropriate solvent with various of the other ingredients enumerated herein, as required, followed by filtered sterilization. The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

### Combination Therapy

In certain aspects, the subject having or at risk for developing pulmonary fibrosis may receive an additional therapy for pulmonary fibrosis. The additional therapy may be administered prior to, simultaneously with, or after administration of the rAAV of the present disclosure. The additional therapy may include administration of an agent, e.g., a compound for treating pulmonary fibrosis. Such an agent may be an agent having an anti-inflammatory and/or an anti-fibrotic property. In certain aspects, the agent may be Perfenidone (Esbriet) or Nintedanib (Ofev).

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method for treating pulmonary fibrosis in a subject, the method comprising administering to the subject a therapeutically effective dose of a recombinant adeno-associated viral (rAAV) virion comprising a nucleic acid encoding sphingosine-1-phosphate lyase (SPL), wherein administering the rAAV virion results in expression of the SPL in the subject thereby treating the pulmonary fibrosis in the subject.

2. A method for preventing pulmonary fibrosis in a subject, the method comprising administering to the subject a therapeutically effective dose of a recombinant adeno-associated viral (rAAV) virion comprising a nucleic acid encoding sphingosine-1-phosphate lyase (SPL), wherein administering the rAAV virion results in expression of the SPL in the subject thereby preventing the pulmonary fibrosis in the subject.

3. The method of aspect 1 or 2, wherein the rAAV virion comprises AAV serotype 9 capsid proteins.

4. The method of aspect 1 or 2, wherein the rAAV virion comprises AAV-PHP.eb virions.

5. The method of aspect 1 or 2, wherein the rAAV virion comprises AAV-PHP.S virions.

6. The method of aspect 1 or 2, wherein the rAAV virion comprises a vector comprising an AAV inverted terminal repeat, a promoter/enhancer, a nucleic acid sequence encoding human SPL, and an AAV inverted terminal repeat, wherein the nucleic acid encoding the human SPL is flanked by the AAV inverted terminal repeats (ITRs).

7. The method of aspect 6, wherein the rAAV vector is AAV-PHP.B, AAV-PHP.eB, AAV-PHP.S or AAV-Anc80.

8. The method of aspect 6, wherein the rAAV vector is AAV-9 vector.

9. The method of any one of aspects 6-8, wherein the promoter is a cytomegalovirus (CMV) promoter, chicken β-actin promoter, human SGPL-1 gene promoter, human β-actin/CMV hybrid promoter, chicken β-actin/CMV hybrid promoter, CMV actin-Globin (CAG) Hybrid Promoter, or albumin promoter.

10. The method of any one of aspects 1-9, wherein the subject is a human and the SPL is a human SPL.

11. The method of any one of aspects 1-10, wherein the administering comprises intravenous administering.

12. The method of any one of aspects 1-11, wherein the administering comprises administering a dose comprising 1011, 5×1011, 1012, 5×1012, 1013, or 5×1013 viral genomes (vg) to the subject.

13. The method of any one of aspects 1-12, wherein the administering comprises administering to the subject an agent having an anti-inflammatory property and/or an anti-fibrotic property.

14. The method of aspect 13, wherein the administering comprises administering Perfenidone (Esbriet) to the subject.

15. The method of aspect 13 or 14, wherein the administering comprises administering Nintedanib (Ofev) to the subject.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, dimensions, etc.) but some experimental errors and deviations should be accounted for.

### Example 1: AAV-SPL Protects Against Bleomycin-Induced Pulmonary Fibrosis

Lung fibrosis was induced in wild type C57BL/6 male mice at eight weeks of age by intratracheal installation of 3 U/kg clinical grade bleomycin (Fresenius Kabi Pharmaceutical) in saline (Bleo) on day 0. Bleomycin-treated mice either received 5×10¹¹ vg AAV-SPL (n=8) or saline (n=6) by tail vein injection on day 14. A control group received only saline by tail vein injection (n=6). Mice were euthanized by CO₂ inhalation and both lungs were harvested. Lung tissue was analyzed for hydroxyproline content using a hydroxyproline test kit (Sigma-Aldrich) according to the manufacturer's instructions. Using Holm-Sidak multiple comparisons test, P=0.0321 for Bleo+AAV-SPL vs. Bleo+Saline.

Human WT SGPL1 cDNA was subcloned into EcoRI/XhoI sites in pAAV-MCS plasmid (Agilent Technologies) in which SGPL1 expression is driven by the CMV promoter/enhancer. The virus was amplified and packaged in AAV9 capsid.

Sequence of pAAV-CMV-hSPL is set forth in SEQ ID NO:4:

(SEQ ID NO: 4)
CCTGCAGGCAGCTGCGCGCTCGCTCGCTCACTGAGGCCGCCCGGGCA
AAGCCCGGGCGTCGGGCGACCTTTGGTCGCCCGGCCTCAGTGAGCGAGCGAGCGCG
CAGAGAGGGAGTGGCCAACTCCATCACTAGGGGTTCCTGCGGCCGCACGCGTGGAG
CTAGTTATTAATAGTAATCAATTACGGGGTCATTAGTTCATAGCCCATATATGGAGT
TCCGCGTTACATAACTTACGGTAAATGGCCCGCCTGGCTGACCGCCCAACGACCCCC
GCCCATTGACGTCAATAATGACGTATGTTCCCATAGTAACGTCAATAGGGACTTTCC
ATTGACGTCAATGGGTGGAGTATTTACGGTAAACTGCCCACTTGGCAGTACATCAA
GTGTATCATATGCCAAGTACGCCCCCTATTGACGTCAATGACGGTAAATGGCCCGCC
TGGCATTATGCCCAGTACATGACCTTATGGGACTTTCCTACTTGGCAGTACATCTAC
GTATTAGTCATCGCTATTACCATGGTGATGCGGTTTTGGCAGTACATCAATGGGCGT
GGATAGCGGTTTGACTCACGGGGATTTCCAAGTCTCCACCCCATTGACGTCAATGGG
AGTTTGTTTTGCACCAAAATCAACGGGACTTTCCAAAATGTCGTAACAACTCCGCCC
CATTGACGCAAATGGGCGGTAGGCGTGTACGGTGGGAGGTCTATATAAGCAGAGCT
CGTTTAGTGAACCGTCAGATCGCCTGGAGACGCCATCCACGCTGTTTTGACCTCCAT
AGAAGACACCGGGACCGATCCAGCCTCCGCGGATTCGAATCCCGGCCGGGAACGGT
GCATTGGAACGCGGATTCCCCGTGCCAAGAGTGACGTAAGTACCGCCTATAGAGTC
TATAGGCCCACAAAAAATGCTTTCTTCTTTTAATATACTTTTTTGTTTATCTTATTTCT
AATACTTTCCCTAATCTCTTTCTTTCAGGGCAATAATGATACAATGTATCATGCCTCT
TTGCACCATTCTAAAGAATAACAGTGATAATTTCTGGGTTAAGGCAATAGCAATATT
TCTGCATATAAATATTTCTGCATATAAATTGTAACTGATGTAAGAGGTTTCATATTG
CTAATAGCAGCTACAATCCAGCTACCATTCTGCTTTTATTTTATGGTTGGGATAAGG
CTGGATTATTCTGAGTCCAAGCTAGGCCCTTTTGCTAATCATGTTCATACCTCTTATC
TTCCTCCCACAGCTCCTGGGCAACGTGCTGGTCTGTGTGCTGGCCCATCACTTTGGC
AAAGAATTGGGATTCGAACATCGATTGAATTCGCCACCATGCCTAGCACAGACCTT
CTGATGTTGAAGGCCTTTGAGCCCTACTTAGAGATTTTGGAAGTATACTCCACAAAA
GCCAAGAATTATGTAAATGGACATTGCACCAAGTATGAGCCCTGGCAGCTAATTGC
ATGGAGTGTCGTGTGGACCCTGCTGATAGTCTGGGGATATGAGTTTGTCTTCCAGCC
AGAGAGTTTATGGTCAAGGTTTAAAAAGAAATGTTTTAAGCTCACCAGGAAGATGC
CCATTATTGGTCGTAAGATTCAAGACAAGTTGAACAAGACCAAGGATGATATTAGC
AAGAACATGTCATTCCTGAAAGTGGACAAAGAGTATGTGAAAGCTTTACCCTCCCA
GGGTCTGAGCTCATCTGCTGTTTTGGAGAAACTTAAGGAGTACAGCTCTATGGACGC
CTTCTGGCAAGAGGGGAGAGCCTCTGGAACAGTGTACAGTGGGGAGGAGAAGCTC
ACTGAGCTCCTTGTGAAGGCTTATGGAGATTTTGCATGGAGTAACCCCCTGCATCCA
GATATCTTCCCAGGACTACGCAAGATAGAGGCAGAAATTGTGAGGATAGCTTGTTC
CCTGTTCAATGGGGGACCAGATTCGTGTGGATGTGTGACTTCTGGGGGAACAGAAA
GCATACTGATGGCCTGCAAAGCATATCGGGATCTGGCCTTTGAGAAGGGGATCAAA
ACTCCAGAAATTGTGGCTCCCCAAAGTGCCCATGCTGCATTTAACAAAGCAGCCAG
TTACTTTGGGATGAAGATTGTGCGGGTCCCATTGACGAAGATGATGGAGGTGGATG
TGCGGGCAATGAGAAGAGCTATCTCCAGGAACACTGCCATGCTCGTCTGTTCTACCC
CACAGTTTCCTCATGGTGTAATAGATCCTGTCCCTGAAGTGGCCAAGCTGGCTGTCA
AATACAAAATACCCCTTCATGTCGACGCTTGTCTGGGAGGCTTCCTCATCGTCTTTA
TGGAGAAAGCAGGATACCCACTGGAGCACCCATTTGATTTCCGGGTGAAAGGTGTA
ACCAGCATTTCAGCTGACACCCATAAGTATGGCTATGCCCCAAAAGGCTCATCATTG
GTGTTGTATAGTGACAAGAAGTACAGGAACTATCAGTTCTTCGTCGATACAGATTG
GCAGGGTGGCATCTATGCTTCCCCAACCATCGCAGGCTCACGGCCTGGTGGCATTA
GCGCAGCCTGTTGGGCTGCCTTGATGCACTTCGGTGAGAACGGCTATGTTGAAGCTA
CCAAACAGATCATCAAAACTGCTCGCTTCCTCAAGTCAGAACTGGAAAATATCAAA
GGCATCTTTGTTTTTGGGAATCCCCAATTGTCAGTCATTGCTCTGGGATCCCGTGATT
TTGACATCTACCGACTATCAAACCTGATGACTGCTAAGGGGTGGAACTTGAACCAG
TTGCAGTTCCCACCCAGTATTCATTTCTGCATCACATTACTACACGCCCGGAAACGA
GTAGCTATACAATTCCTAAAGGACATTCGAGAATCTGTCACTCAAATCATGAAGAA
TCCTAAAGCGAAGACCACAGGAATGGGTGCCATCTATGGCATGGCCCAGACAACTG
TTGACAGGAATATGGTTGCAGAATTGTCCTCAGTCTTCTTGGACAGCTTGTACAGCA
CCGACACTGTCACCCAGGGCAGCCAGATGAATGGTTCTCCAAAACCCCACTGACTC
GAGAGATCTACGGGTGGCATCCCTGTGACCCCTCCCCAGTGCCTCTCCTGGCCCTGG
AAGTTGCCACTCCAGTGCCCACCAGCCTTGTCCTAATAAAATTAAGTTGCATCATTT
TGTCTGACTAGGTGTCCTTCTATAATATTATGGGGTGGAGGGGGGTGGTATGGAGC
AAGGGGCAAGTTGGGAAGACAACCTGTAGGGCCTGCGGGGTCTATTGGGAACCAA
GCTGGAGTGCAGTGGCACAATCTTGGCTCACTGCAATCTCCGCCTCCTGGGTTCAAG
CGATTCTCCTGCCTCAGCCTCCCGAGTTGTTGGGATTCCAGGCATGCATGACCAGGC
TCAGCTAATTTTTGTTTTTTTGGTAGAGACGGGGTTTCACCATATTGGCCAGGCTGG
TCTCCAACTCCTAATCTCAGGTGATCTACCCACCTTGGCCTCCCAAATTGCTGGGAT
TACAGGCGTGAACCACTGCTCCCTTCCCTGTCCTTCTGATTTTGTAGGTAACCACGT
GCGGACCGAGCGGCCGCAGGAACCCCTAGTGATGGAGTTGGCCACTCCCTCTCTGC
GCGCTCGCTCGCTCACTGAGGCCGGGCGACCAAAGGTCGCCCGACGCCCGGGCTTT
GCCCGGGCGGCCTCAGTGAGCGAGCGAGCGCGCAGCTGCCTGCAGGGGCGCCTGAT
GCGGTATTTTCTCCTTACGCATCTGTGCGGTATTTCACACCGCATACGTCAAAGCAA
CCATAGTACGCGCCCTGTAGCGGCGCATTAAGCGCGGCGGGTGTGGTGGTTACGCG
CAGCGTGACCGCTACACTTGCCAGCGCCCTAGCGCCCGCTCCTTTCGCTTTCTTCCCT
TCCTTTCTCGCCACGTTCGCCGGCTTTCCCCGTCAAGCTCTAAATCGGGGGCTCCCTT
TAGGGTTCCGATTTAGTGCTTTACGGCACCTCGACCCCAAAAAACTTGATTTGGGTG
ATGGTTCACGTAGTGGGCCATCGCCCTGATAGACGGTTTTTCGCCCTTTGACGTTGG
AGTCCACGTTCTTTAATAGTGGACTCTTGTTCCAAACTGGAACAACACTCAACCCTA
TCTCGGGCTATTCTTTTGATTTATAAGGGATTTTGCCGATTTCGGCCTATTGGTTAAA
AAATGAGCTGATTTAACAAAAATTTAACGCGAATTTTAACAAAATATTAACGTTTAC
AATTTTATGGTGCACTCTCAGTACAATCTGCTCTGATGCCGCATAGTTAAGCCAGCC
CCGACACCCGCCAACACCCGCTGACGCGCCCTGACGGGCTTGTCTGCTCCCGGCATC
CGCTTACAGACAAGCTGTGACCGTCTCCGGGAGCTGCATGTGTCAGAGGTTTTCACC
GTCATCACCGAAACGCGCGAGACGAAAGGGCCTCGTGATACGCCTATTTTTATAGG
TTAATGTCATGATAATAATGGTTTCTTAGACGTCAGGTGGCACTTTTCGGGGAAATG
TGCGCGGAACCCCTATTTGTTTATTTTTCTAAATACATTCAAATATGTATCCGCTCAT
GAGACAATAACCCTGATAAATGCTTCAATAATATTGAAAAAGGAAGAGTATGAGTA
TTCAACATTTCCGTGTCGCCCTTATTCCCTTTTTTGCGGCATTTTGCCTTCCTGTTTTT
GCTCACCCAGAAACGCTGGTGAAAGTAAAAGATGCTGAAGATCAGTTGGGTGCACG
AGTGGGTTACATCGAACTGGATCTCAACAGCGGTAAGATCCTTGAGAGTTTTCGCCC
CGAAGAACGTTTTCCAATGATGAGCACTTTTAAAGTTCTGCTATGTGGCGCGGTATT
ATCCCGTATTGACGCCGGGCAAGAGCAACTCGGTCGCCGCATACACTATTCTCAGA
ATGACTTGGTTGAGTACTCACCAGTCACAGAAAAGCATCTTACGGATGGCATGACA
GTAAGAGAATTATGCAGTGCTGCCATAACCATGAGTGATAACACTGCGGCCAACTT
ACTTCTGACAACGATCGGAGGACCGAAGGAGCTAACCGCTTTTTTGCACAACATGG
GGGATCATGTAACTCGCCTTGATCGTTGGGAACCGGAGCTGAATGAAGCCATACCA
AACGACGAGCGTGACACCACGATGCCTGTAGCAATGGCAACAACGTTGCGCAAACT
ATTAACTGGCGAACTACTTACTCTAGCTTCCCGGCAACAATTAATAGACTGGATGGA
GGCGGATAAAGTTGCAGGACCACTTCTGCGCTCGGCCCTTCCGGCTGGCTGGTTTAT
TGCTGATAAATCTGGAGCCGGTGAGCGTGGGTCTCGCGGTATCATTGCAGCACTGG
GGCCAGATGGTAAGCCCTCCCGTATCGTAGTTATCTACACGACGGGGAGTCAGGCA
ACTATGGATGAACGAAATAGACAGATCGCTGAGATAGGTGCCTCACTGATTAAGCA
TTGGTAACTGTCAGACCAAGTTTACTCATATATACTTTAGATTGATTTAAAACTTCA
TTTTTAATTTAAAAGGATCTAGGTGAAGATCCTTTTTGATAATCTCATGACCAAAAT
CCCTTAACGTGAGTTTTCGTTCCACTGAGCGTCAGACCCCGTAGAAAAGATCAAAG
GATCTTCTTGAGATCCTTTTTTTCTGCGCGTAATCTGCTGCTTGCAAACAAAAAAAC
CACCGCTACCAGCGGTGGTTTGTTTGCCGGATCAAGAGCTACCAACTCTTTTTCCGA
AGGTAACTGGCTTCAGCAGAGCGCAGATACCAAATACTGTCCTTCTAGTGTAGCCG
TAGTTAGGCCACCACTTCAAGAACTCTGTAGCACCGCCTACATACCTCGCTCTGCTA
ATCCTGTTACCAGTGGCTGCTGCCAGTGGCGATAAGTCGTGTCTTACCGGGTTGGAC
TCAAGACGATAGTTACCGGATAAGGCGCAGCGGTCGGGCTGAACGGGGGGTTCGTG
CACACAGCCCAGCTTGGAGCGAACGACCTACACCGAACTGAGATACCTACAGCGTG
AGCTATGAGAAAGCGCCACGCTTCCCGAAGGGAGAAAGGCGGACAGGTATCCGGT
AAGCGGCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCGATTTTTGT
GATGCTCGTCAGGGGGGCGGAGCCTATGGAAAAACGCCAGCAACGCGGCCTTTTTA
CGGTTCCTGGCCTTTTGCTGGCCTTTTGCTCACATGT.

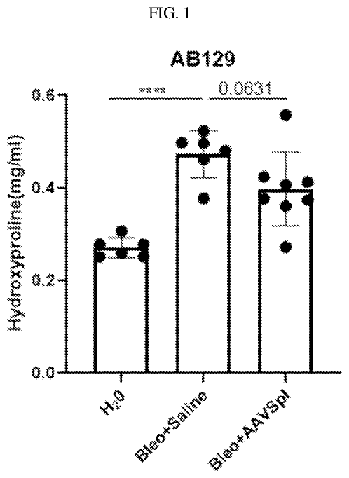

FIG. 1. AAV-SPL protects against bleomycin-induced pulmonary fibrosis. CMV promoter/enhancer is driving SPL expression. Reduced hydroxyproline level (a common measure of collagen) is an indicator of reduced lung fibrosis. Reduced picrosirius red staining (another indicator of fibrosis) was also observed (data not shown).

A modified version of AAV-SPL referred to as AAV-SPL 2.0, in which SGPL1 expression is driven by the synthetic “CAG” promoter has been created by replacing the tomato gene in pAAV-CAG-dtTomato (Addgene #59462). This vector will be used for further experiments.

pAAV-CAG-hSPL sequence is set forth in SEQ ID NO:5.

(SEQ ID NO: 5)
CTGCGCGCTCGCTCGCTCACTGAGGCCGCCCGGGCAAAGCCCGGGCG
TCGGGCGACCTTTGGTCGCCCGGCCTCAGTGAGCGAGCGAGCGCGCAGAGAGGGAG
TGGCCAACTCCATCACTAGGGGTTCCTTGTAGTTAATGATTAACCCGCCATGCTACT
TATCTACGTAGCCATGCTCTAGGAAGATCGTACCATTGACGTCAATAATGACGTATG
TTCCCATAGTAACGCCAATAGGGACTTTCCATTGACGTCAATGGGTGGAGTATTTAC
GGTAAACTGCCCACTTGGCAGTACATCAAGTGTATCATATGCCAAGTACGCCCCCTA
TTGACGTCAATGACGGTAAATGGCCCGCCTGGCATTATGCCCAGTACATGACCTTAT
GGGACTTTCCTACTTGGCAGTACATCTACGTATTAGTCATCGCTATTACCATGGTCG
AGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGG
GGGGCGCGCGCCAGGCGGGGGGGGGCGGGGCGAGGGGCGGGGCGGGGCGAGGCG
GAGAGGTGCGGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGGGCGGGAGT
CGCTGCGACGCTGCCTTCGCCCCGTGCCCCGCTCCGCCGCCGCCTCGCGCCGCCCGC
CCCGGCTCTGACTGACCGCGTTACTCCCACAGGTGAGCGGGCGGGACGGCCCTTCT
CCTCCGGGCTGTAATTAGCGCTTGGTTTAATGACGGCTTGTTTCTTTTCTGTGGCTGC
GTGAAAGCCTTGAGGGGCTCCGGGAGGGCCCTTTGTGCGGGGGGAGCGGCTCGGGG
CTGTCCGCGGGGGGACGGCTGCCTTCGGGGGGGACGGGGCAGGGCGGGGTTCGGCT
TCTGGCGTGTGACCGGCGGCTCTAGAGCCTCTGCTAACCATGTTCATGCCTTCTTCTT
TTTCCTACAGCTCCTGGGCAACGTGCTGGTTATTGTGCTGTCTCATCATTTTGGCAAA
GAATTGGATCCGGTACCGCCACCATGGCGAGCACAGACCTTCTGATGTTGAAGGCC
TTTGAGCCCTACTTAGAGATTTTGGAAGTATACTCCACAAAAGCCAAGAATTATGTA
AATGGACATTGCACCAAGTATGAGCCCTGGCAGCTAATTGCATGGAGTGTCGTGTG
GACCCTGCTGATAGTCTGGGGATATGAGTTTGTCTTCCAGCCAGAGAGTTTATGGTC
AAGGTTTAAAAAGAAATGTTTTAAGCTCACCAGGAAGATGCCCATTATTGGTCGTA
AGATTCAAGACAAGTTGAACAAGACCAAGGATGATATTAGCAAGAACATGTCATTC
CTGAAAGTGGACAAAGAGTATGTGAAAGCTTTACCCTCCCAGGGTCTGAGCTCATC
TGCTGTTTTGGAGAAACTTAAGGAGTACAGCTCTATGGACGCCTTCTGGCAAGAGG
GGAGAGCCTCTGGAACAGTGTACAGTGGGGAGGAGAAGCTCACTGAGCTCCTTGTG
AAGGCTTATGGAGATTTTGCATGGAGTAACCCCCTGCATCCAGATATCTTCCCAGGA
CTACGCAAGATAGAGGCAGAAATCGTGAGGATAGCTTGTTCCCTGTTCAATGGGGG
ACCAGATTCGTGTGGATGTGTGACTTCTGGGGGAACAGAAAGCATACTGATGGCCT
GCAAAGCATATCGGGATCTGGCCTTTGAGAAGGGGATCAAAACTCCAGAAATTGTG
GCTCCCCAAAGTGCCCATGCTGCATTTAACAAAGCAGCCAGTTACTTTGGGATGAA
GATTGTGCGGGTCCCATTGACGAAGATGATGGAGGTGGATGTGCGGGCAATGAGAA
GAGCTATCTCCAGGAACACTGCCATGCTCGTCTGTTCTACCCCACAGTTTCCTCATG
GTGTAATAGATCCTGTCCCTGAAGTGGCCAAGCTGGCTGTCAAATACAAAATACCC
CTTCATGTCGACGCTTGTCTGGGAGGCTTCCTCATCGTCTTTATGGAGAAAGCAGGA
TACCCACTGGAGCACCCATTTGATTTCCGGGTGAAAGGTGTAACCAGCATTTCAGCT
GACACCCATAAGTATGGCTATGCCCCAAAAGGCTCATCATTGGTGTTGTATAGTGAC
AAGAAGTACAGGAACTATCAGTTCTTCGTCGATACAGATTGGCAGGGTGGCATCTA
TGCTTCCCCAACCATCGCAGGCTCACGGCCTGGTGGCATTAGCGCAGCCTGTTGGGC
TGCCTTGATGCACTTCGGTGAGAACGGCTATGTTGAAGCTACCAAACAGATCATCA
AAACTGCTCGCTTCCTCAAGTCAGAACTGGAAAATATCAAAGGCATCTTTGTTTTTG
GGAATCCCCAATTGTCAGTCATTGCTCTGGGATCCCGTGATTTTGACATCTACCGAC
TATCAAACCTGATGACTGCTAAGGGGTGGAACTTGAACCAGTTGCAGTTCCCACCC
AGTATTCATTTCTGCATCACATTACTACACGCCCGGAAACGAGTAGCTATACAATTC
CTAAAGGACATTCGAGAATCTGTCACTCAAATCATGAAGAATCCTAAAGCGAAGAC
CACAGGAATGGGTGCCATCTATGGCATGGCCCAGACAACTGTTGACAGGAATATGG
TTGCAGAATTGTCCTCAGTCTTCTTGGACAGCTTGTACAGCACCGACACTGTCACCC
AGGGCAGCCAGATGAATGGTTCTCCAAAACCCCACTGAGAATTCGATATCAAGCTT
ATCGATAATCAACCTCTGGATTACAAAATTTGTGAAAGATTGACTGGTATTCTTAAC
TATGTTGCTCCTTTTACGCTATGTGGATACGCTGCTTTAATGCCTTTGTATCATGCTA
TTGCTTCCCGTATGGCTTTCATTTTCTCCTCCTTGTATAAATCCTGGTTGCTGTCTCTT
TATGAGGAGTTGTGGCCCGTTGTCAGGCAACGTGGCGTGGTGTGCACTGTGTTTGCT
GACGCAACCCCCACTGGTTGGGGCATTGCCACCACCTGTCAGCTCCTTTCCGGGACT
TTCGCTTTCCCCCTCCCTATTGCCACGGCGGAACTCATCGCCGCCTGCCTTGCCCGCT
GCTGGACAGGGGCTCGGCTGTTGGGCACTGACAATTCCGTGGTGTTGTCGGGGAAA
TCATCGTCCTTTCCTTGGCTGCTCGCCTGTGTTGCCACCTGGATTCTGCGCGGGACGT
CCTTCTGCTACGTCCCTTCGGCCCTCAATCCAGCGGACCTTCCTTCCCGCGGCCTGCT
GCCGGCTCTGCGGCCTCTTCCGCGTCTTCGCCTTCGCCCTCAGACGAGTCGGATCTC
CCTTTGGGCCGCCTCCCCGCATCGATACCGTCGACCCGGGCGGCCGCTTCGAGCAG
ACATGATAAGATACATTGATGAGTTTGGACAAACCACAACTAGAATGCAGTGAAAA
AAATGCTTTATTTGTGAAATTTGTGATGCTATTGCTTTATTTGTAACCATTATAAGCT
GCAATAAACAAGTTAACAACAACAATTGCATTCATTTTATGTTTCAGGTTCAGGGGG
AGATGTGGGAGGTTTTTTAAAGCAAGTAAAACCTCTACAAATGTGGTAAAATCGAT
AAGGATCTTCCTAGAGCATGGCTACGTAGATAAGTAGCATGGCGGGTTAATCATTA
ACTACAAGGAACCCCTAGTGATGGAGTTGGCCACTCCCTCTCTGCGCGCTCGCTCGC
TCACTGAGGCCGGGCGACCAAAGGTCGCCCGACGCCCGGGCTTTGCCCGGGCGGCC
TCAGTGAGCGAGCGAGCGCGCAGCTGCATTAATGAATCGGCCAACGCGCGGGGAG
AGGCGGTTTGCGTATTGGGCGCTCTTCCGCTTCCTCGCTCACTGACTCGCTGCGCTC
GGTCGTTCGGCTGCGGCGAGCGGTATCAGCTCACTCAAAGGCGGTAATACGGTTAT
CCACAGAATCAGGGGATAACGCAGGAAAGAACATGTGAGCAAAAGGCCAGCAAAA
GGCCAGGAACCGTAAAAAGGCCGCGTTGCTGGCGTTTTTCCATAGGCTCCGCCCCC
CTGACGAGCATCACAAAAATCGACGCTCAAGTCAGAGGTGGCGAAACCCGACAGG
ACTATAAAGATACCAGGCGTTTCCCCCTGGAAGCTCCCTCGTGCGCTCTCCTGTTCC
GACCCTGCCGCTTACCGGATACCTGTCCGCCTTTCTCCCTTCGGGAAGCGTGGCGCT
TTCTCATAGCTCACGCTGTAGGTATCTCAGTTCGGTGTAGGTCGTTCGCTCCAAGCT
GGGCTGTGTGCACGAACCCCCCGTTCAGCCCGACCGCTGCGCCTTATCCGGTAACTA
TCGTCTTGAGTCCAACCCGGTAAGACACGACTTATCGCCACTGGCAGCAGCCACTG
GTAACAGGATTAGCAGAGCGAGGTATGTAGGCGGTGCTACAGAGTTCTTGAAGTGG
TGGCCTAACTACGGCTACACTAGAAGAACAGTATTTGGTATCTGCGCTCTGCTGAAG
CCAGTTACCTTCGGAAAAAGAGTTGGTAGCTCTTGATCCGGCAAACAAACCACCGC
TGGTAGCGGTGGTTTTTTTGTTTGCAAGCAGCAGATTACGCGCAGAAAAAAAGGAT
CTCAAGAAGATCCTTTGATCTTTTCTACGGGGTCTGACGCTCAGTGGAACGAAAACT
CACGTTAAGGGATTTTGGTCATGAGATTATCAAAAAGGATCTTCACCTAGATCCTTT
TAAATTAAAAATGAAGTTTTAAATCAATCTAAAGTATATATGAGTAAACTTGGTCTG
ACAGTTACCAATGCTTAATCAGTGAGGCACCTATCTCAGCGATCTGTCTATTTCGTT
CATCCATAGTTGCCTGACTCCCCGTCGTGTAGATAACTACGATACGGGAGGGCTTAC
CATCTGGCCCCAGTGCTGCAATGATACCGCGAGACCCACGCTCACCGGCTCCAGAT
TTATCAGCAATAAACCAGCCAGCCGGAAGGGCCGAGCGCAGAAGTGGTCCTGCAAC
TTTATCCGCCTCCATCCAGTCTATTAATTGTTGCCGGGAAGCTAGAGTAAGTAGTTC
GCCAGTTAATAGTTTGCGCAACGTTGTTGCCATTGCTACAGGCATCGTGGTGTCACG
CTCGTCGTTTGGTATGGCTTCATTCAGCTCCGGTTCCCAACGATCAAGGCGAGTTAC
ATGATCCCCCATGTTGTGCAAAAAAGCGGTTAGCTCCTTCGGTCCTCCGATCGTTGT
CAGAAGTAAGTTGGCCGCAGTGTTATCACTCATGGTTATGGCAGCACTGCATAATTC
TCTTACTGTCATGCCATCCGTAAGATGCTTTTCTGTGACTGGTGAGTACTCAACCAA
GTCATTCTGAGAATAGTGTATGCGGCGACCGAGTTGCTCTTGCCCGGCGTCAATACG
GGATAATACCGCGCCACATAGCAGAACTTTAAAAGTGCTCATCATTGGAAAACGTT
CTTCGGGGCGAAAACTCTCAAGGATCTTACCGCTGTTGAGATCCAGTTCGATGTAAC
CCACTCGTGCACCCAACTGATCTTCAGCATCTTTTACTTTCACCAGCGTTTCTGGGTG
AGCAAAAACAGGAAGGCAAAATGCCGCAAAAAAGGGAATAAGGGCGACACGGAA
ATGTTGAATACTCATACTCTTCCTTTTTCAATATTATTGAAGCATTTATCAGGGTTAT
TGTCTCATGAGCGGATACATATTTGAATGTATTTAGAAAAATAAACAAATAGGGGT
TCCGCGCACATTTCCCCGAAAAGTGCCACCTAAATTGTAAGCGTTAATATTTTGTTA
AAATTCGCGTTAAATTTTTGTTAAATCAGCTCATTTTTTAACCAATAGGCCGAAATC
GGCAAAATCCCTTATAAATCAAAAGAATAGACCGAGATAGGGTTGAGTGTTGTTCC
AGTTTGGAACAAGAGTCCACTATTAAAGAACGTGGACTCCAACGTCAAAGGGCGAA
AAACCGTCTATCAGGGCGATGGCCCACTACGTGAACCATCACCCTAATCAAGTTTTT
TGGGGTCGAGGTGCCGTAAAGCACTAAATCGGAACCCTAAAGGGAGCCCCCGATTT
AGAGCTTGACGGGGAAAGCCGGCGAACGTGGCGAGAAAGGAAGGGAAGAAAGCG
AAAGGAGCGGGCGCTAGGGCGCTGGCAAGTGTAGCGGTCACGCTGCGCGTAACCA
CCACACCCGCCGCGCTTAATGCGCCGCTACAGGGCGCGTCCCATTCGCCATTCAGGC
TGCGCAACTGTTGGGAAGGGCGATCGGTGCGGGCCTCTTCGCTATTACGCCAG.

### Example 2: Optimization of AAV-SPL Delivery

AAV-SPL may be delivered via the intratracheal route, which may be more effective and less toxic. Intratracheal delivery is expected to result in 100-1000-fold higher AAV9 transduction in lung than in other tissues. The effects of 5e11 vector genomes of AAV-SPL given by intravenous vs intratracheal routes will be compared in wild type mice treated with intratracheal bleomycin to induce pulmonary fibrosis. This is the most widely used murine model due to similarity to human pulmonary fibrosis, ease of induction, and reproducibility. The optimal delivery route will be used to conduct a dose response.

### Example 3: Mechanism of Action of AAV-SPL

AAV-SPL may transduce lung alveolar epithelial cells and lower lung S1P levels, thereby reducing pro-fibrotic (and pro-inflammatory) cytokines. The lung cells transduced by AAV-SPL can be identified using immunofluorescence microscopy to colocalize SPL with cell-specific markers of alveolar epithelial cells, macrophages, fibroblasts in the bleomycin mouse model. The impact of AAV-SPL on lung S1P levels can be measured in the bleomycin model. The impact of AAV-SPL on key pro-fibrotic and pro-inflammatory cytokines and signaling pathways can be measured in the bleomycin mouse model.

### Example 4: Validation in Additional Genetic Model

The effect of AAV-SPL will be tested in Trf1 knockout mice. In this cre/lox model, lung fibrosis is driven by shortened telomeres in type 2 airway epithelial cells, emulating the shortened telomeres observed in the type 2 airway epithelial cells of idiopathic pulmonary fibrosis patients. Survival, lung pathology, cytokines and S1P levels will serve as endpoints.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method for treating pulmonary fibrosis in a subject, the method comprising administering to the subject a therapeutically effective dose of a recombinant adeno-associated viral (rAAV) virion comprising a nucleic acid encoding sphingosine-1-phosphate lyase (SPL), wherein administering the rAAV virion results in expression of the SPL in the subject thereby treating the pulmonary fibrosis in the subject.

2. A method for preventing pulmonary fibrosis in a subject, the method comprising administering to the subject a therapeutically effective dose of a recombinant adeno-associated viral (rAAV) virion comprising a nucleic acid encoding sphingosine-1-phosphate lyase (SPL), wherein administering the rAAV virion results in expression of the SPL in the subject thereby preventing the pulmonary fibrosis in the subject.

3. The method of claim 1, wherein the rAAV virion comprises AAV serotype 9 capsid proteins.

4. The method of claim 1, wherein the rAAV virion comprises AAV-PHP.eb virions.

5. The method of claim 1, wherein the rAAV virion comprises AAV-PHP.S virions.

6. The method of claim 1, wherein the rAAV virion comprises a vector comprising an AAV inverted terminal repeat, a promoter/enhancer, a nucleic acid sequence encoding human SPL, and an AAV inverted terminal repeat, wherein the nucleic acid encoding the human SPL is flanked by the AAV inverted terminal repeats (ITRs).

7. The method of claim 6, wherein the rAAV vector is AAV-PHP.B, AAV-PHP.eB, AAV-PHP.S or AAV-Anc80.

8. The method of claim 6, wherein the rAAV vector is AAV-9 vector.

9. The method of claim 6, wherein the promoter is a cytomegalovirus (CMV) promoter, chicken β-actin promoter, human SGPL-1 gene promoter, human β-actin/CMV hybrid promoter, chicken β-actin/CMV hybrid promoter, CMV actin-Globin (CAG) Hybrid Promoter, or albumin promoter.

10. The method of claim 1, wherein the subject is a human and the SPL is a human SPL.

11. The method of claim 1, wherein the administering comprises intravenous administering.

12. The method of claim 1, wherein the administering comprises administering a dose comprising 10¹¹, 5×10¹¹, 10¹², 5×10¹², 10¹³, or 5×10¹³ viral genomes (vg) to the subject.

13. The method of claim 1, wherein the administering comprises administering to the subject an agent having an anti-inflammatory property and/or an anti-fibrotic property.

14. The method of claim 13, wherein the administering comprises administering Perfenidone (Esbriet) to the subject.

15. The method of claim 13, wherein the administering comprises administering Nintedanib (Ofev) to the subject.
